(19) United States
(12) Patent Application Publication
(10) Pub. No.: US 2026/0325574 A1
(43) Pub. Date: Oct. 22, 2026

(54) SODIUM BICARBONATE NUTRITIONAL SUPPLEMENT

(71) Applicant: Laminaria Group AB, Göteborg (SE)

(72) Inventor: Martin Ahnoff, Göteborg (SE)

(21) Appl. No.: 19/727,428

(22) Filed: Jul. 1, 2026

Related U.S. Application Data
(63) Continuation of application No. 17/893,880, filed on Aug. 23, 2022.

(30) Foreign Application Priority Data
Aug. 24, 2021 (EP) 21192881.7
Jun. 1, 2022 (EP) 22176761.9

(51) Int. Cl. A61K 33/00; A61K 9/06; A61K 9/16; A61P 7/08

(52) U.S. Cl. CPC A61K 33/00; A61K 9/06; A61K 9/1652; A61P 7/08

(57) ABSTRACT

An ingestible particle comprising sodium bicarbonate, wherein the particle has size in the range of more than 1.0 mm but not more than 5.0 mm with a thickness in at least one dimension of more than 1.0 mm but not more than 2.0 mm; and wherein the particle contains more than 50% (w/w) of the sodium bicarbonate. The ingestible particles are comprised in a nutritional supplement composition which is a suspension comprising the ingestible particles dispersed in an aqueous liquid, preferably a viscous aqueous medium.

### CROSS-REFERENCE TO RELATED APPLICATIONS

This application is a continuation of U.S. application Ser. No. 17/893,880, filed Aug. 23, 2022, which application claims the benefit of priority to European Application No. EP21192881.7, filed on Aug. 24, 2021, and European Application No. EP22176761.9, filed on Jun. 1, 2022, all of which applications are incorporated by reference herein.

### TECHNICAL FIELD OF THE INVENTION

The present invention relates to the use of sodium bicarbonate as a nutritional supplement for preventing, alleviating or mitigating exercise-induced acidosis and for the improvement of exercise performance. More specifically, the invention relates to improved dosage forms for sodium bicarbonate, which are useful as nutritional supplements for preventing, alleviating or mitigating exercise-induced acidosis and for the improvement of exercise performance. After ingestion, the nutritional supplements release bicarbonate in the blood stream, where the bicarbonate functions as an extracellular buffering agent for the improvement of exercise performance.

The invention further relates to improved dosage forms for sodium bicarbonate, which are useful in pharmaceutical compositions for treating metabolic acidosis.

### BACKGROUND ART

Various nutritional supplements are known to be useful for athletes, see e.g. WO 2019/185742 A1 and EP 2 098 124 A1 which disclose hydrogels. WO 2020/237340 A1 discloses a high-energy food supplement based on inverted brown sugars.

Sodium bicarbonate (NaHCO₃) is a well-established nutritional ergogenic aid. Supplementation with sodium bicarbonate can improve high-intensity exercise performance by augmenting the body's extracellular buffering capacity via an increase in bicarbonate concentration, with various analyses confirming its efficacy (Hadzic et al. The Impact of Sodium Bicarbonate on Performance in Response to Exercise Duration in Athletes: A Systematic Review. 2019. J Sports Sci Med 18: 271-281). Blood bicarbonate is part of the acid-base homeostatic bicarbonate buffer system, which is critical in regulating blood pH concentrations and supporting metabolic functions. As an extracellular buffering agent, NaHCO₃ enhances endogenous bicarbonate buffering capacity by inducing significant elevations in extracellular bicarbonate. Consequently, this enhances efflux of hydrogen cations (H⁺) from skeletal muscle, therefore delaying muscle fatigue and positively affecting numerous performance variables, such as power output and time to exhaustion.

Supplementation with sodium bicarbonate can be useful to prevent, alleviate or mitigate exercise-induced acidosis, i.e. an imbalance in the body's acid-base balance caused by exercise. Exercise-induced acidosis can lead to acidemia, which is defined as arterial blood pH that is lower than 7.35. Supplementation with sodium bicarbonate can also be useful to prevent, alleviate or mitigate exercise-induced acidemia.

Supplementation with sodium bicarbonate can also be useful to treat, including prevent, alleviate or mitigate, metabolic acidosis, i.e. a medical condition characterized by an imbalance in the body's acid-base balance Metabolic acidosis, characterized by blood plasma bicarbonate levels below 22 mmol/l and blood pH below 7.35, can be acute and temporary, as when caused by intense physical exercise, or chronic, as when caused by impaired kidney function (chronic kidney disease, KCD). While acute acidosis will result in temporary impairment of muscle function, chronic acidosis is connected with increased mortality (Adamczak M et al.: Diagnosis and Treatment of Metabolic Acidosis in Patients with Chronic Kidney Disease—Position Statement of the Working Group of the Polish Society of Nephrology. Kidney Blood Press Res 2018; 43:959-969. doi: 10.1159/000490475). Metabolic acidosis can lead to acidemia, which is defined as arterial blood pH that is lower than 7.35. Supplementation with sodium bicarbonate can also be useful to treat, including prevent, alleviate or mitigate, metabolic acidemia.

US 2007/0218126 A1 discloses a composition for reducing inflammation and pain associated with acidosis comprising inter alia calcium carbonate. The two-layered particles have a size of approximately 3 micrometers.

Another benefit of sodium bicarbonate could be the improved recovery from high-intensity exercise.

Substantial changes (~6 mmol/l) in blood bicarbonate improve the likelihood of performance-enhancing effects (Heibel et al. Time to optimize supplementation: modifying factors influencing the individual responses to extracellular buffering agents. 2018. Front Nutr 5: 35). Consequently, large oral doses (0.2-0.3 g/kg body mass) are desirable to induce performance-enhancing elevations in the blood bicarbonate levels.

However, acute gastrointestinal (GI) distress is a known side-effect of ingesting large amounts of NaHCO₃(Burke & Pyne. 2007. Bicarbonate loading to enhance training and competitive performance. Int S Sports Physiol Perform 2: 93-97), particularly when administered as an aqueous solution (Carr et al. 2011. Effect of sodium bicarbonate on [HCO³⁻], pH, and gastrointestinal symptoms. Int J Sport Nutr Exerc Metab 21: 189-194].

A recent study [Middlebrook et al. 2021. Capsule size alters the timing of metabolic alkalosis following sodium bicarbonate supplementation. Frontiers Nutr 8: 27] investigating the effects of NaHCO₃ supplementation using large (5.6 mm or larger) capsules reports some effects on acid-base responses but no effects on GI symptoms and palatability.

Furthermore, GI distress may deter individuals from using NaHCO₃ regardless of its potential ergogenic benefits (Heibel supra).

In conclusion, GI distress such as nausea, vomiting and diarrhoea presents a major practical limitation for athletes' use of NaHCO₃.

It has been suggested that gastro-resistant capsules may alleviate symptoms that are typical with NaHCO₃ingestion (de Oliveira et al. 2018. Is bypassing the stomach a means to optimize sodium bicarbonate supplementation? A case study with a postbariatric surgery individual. Int J Sport Nutr Exerc Metab 26: 1-4). This was to some extent supported by Hilton et al. 2019. A novel ingestion strategy for sodium bicarbonate supplementation in a delayed-release form: a randomised crossover study in trained males. Sports Med 5: 4, who reported lower incidences of GI symptoms, but also an unwanted delay in the time to peak blood [HCO³⁻] and pH.

In addition, enteric-coating has been suggested as a means to reduce gastric symptoms [Hilton et al. 2020. Enteric-Coated Sodium Bicarbonate Attenuates Gastrointestinal Side-Effects. Int J Sport Nutr Exerc Metab 30: 62-68]. Results showed reduction in GI symptoms, but failed to demonstrate the desired increase in blood [HCO₃⁻] and pH.

Furthermore, efficient gastroresistant coatings, widely researched and used in pharmaceuticals, employ enteric polymers which are not regarded as natural ingredients or do not possess GRAS (generally regarded as safe) status by the regulatory bodies, and cannot thus be used for nutritional products like NaHCO₃(Barbosa et al. 2017. Going natural: using polymers from nature for gastroresistant applications. Br J Pharm 2: 14-30].

US 2013/0236545 A1 discloses an oral pharmaceutical formulation which is useful for the treatment of cystinurea, when administered together with another formulation containing a Krebs cycle precursor salt. The formulation is a two-layered mini-tablet consisting of a core including at least bicarbonate salt and at least one prolonged-release matrix, and of a coating including at least one coating agent to ensure a prolonged release.

U.S. Pat. No. 6,432,450 B1 and CN 109430669 A disclose effervescent compositions, i.e. compositions which are designed for releasing the bicarbonate salt within seconds in water prior to ingestion, thereby creating carbon dioxide already prior to ingestion.

RU 2550927 C2 discloses a cough medicament with granules containing sodium bicarbonate for reducing the viscosity of sputum. This implies that the sodium bicarbonate is released already in the oral cavity, again within seconds in water.

Accordingly, there is a need for formulations and methods for nutritional intake of sodium bicarbonate, NaHCO₃, which are associated with a significant reduction in GI side effects following intake.

### SUMMARY OF THE INVENTION

It is an object of the present invention to provide formulations and methods for nutritional intake of sodium bicarbonate, NaHCO₃, which are associated with a significant reduction in GI side effects following intake.

It is a further object to provide formulations and methods for nutritional intake of sodium bicarbonate, which at the same time are associated with high palatability and high gastrointestinal uptake.

It is a further object to provide formulations and methods for nutritional intake of sodium bicarbonate, which facilitate the intake of sufficient amounts of sodium bicarbonate to improve high-intensity exercise performance.

It is a further object to provide formulations and methods for nutritional intake of sodium bicarbonate, which are useful against exercise-induced acidosis or acidemia.

For these and other objects which will be evident from this disclosure, the present invention provides according to a first aspect a nutritional supplement composition which is a suspension comprising ingestible particles dispersed in a viscous aqueous medium.

The viscosity of the aqueous medium prevents or delays sedimentation of the ingestible particles when dispersed in the viscous aqueous medium until the dispersion has been ingested.

The ingestible particle is comprising sodium bicarbonate, wherein the particle has size in the range of more than 1.0 mm but not more than 5.0 mm, with a thickness in at least one dimension of more than 1.0 mm but not more than 2.0 mm; and wherein the particle contains more than 50% (w/w) of the sodium bicarbonate.

The inventors have realized and demonstrated that ingestible particles with these dimensions achieve an efficient uptake of sodium bicarbonate, NaHCO₃, but with a low occurrence of GI side effects. The particles facilitate ingestion and uptake of sodium bicarbonate salt in amounts in the range of 0.20-0.30 g sodium bicarbonate salt per kg of body weight or higher, which is necessary to achieve a substantial change (~4-6 mmol/l) in blood bicarbonate that improves the likelihood of performance-enhancing effects.

The inventors have realized and demonstrated that the ingestible particles achieve an efficient uptake of sodium bicarbonate, but with a low occurrence of GI side effects, in particular when dispersed in a viscous medium, such as a gel or a fluid with viscoelastic properties or a viscous liquid.

According to a second aspect, there is provided a kit for preparing a nutritional supplement composition, the kit comprising ingestible particles as defined herein; and an aqueous medium as defined herein.

According to a further aspect, the nutritional supplement composition comprising the ingestible particles and the kit are useful to improve high-intensity exercise performance. The ingestible particles are useful in the nutritional supplement composition to improve high-intensity exercise performance.

According to a further aspect, there is provided a method for improving high-intensity exercise performance, comprising the step of ingesting the nutritional supplement composition comprising the ingestible particles.

According to one further aspect, the nutritional supplement composition comprising the ingestible particles and the kit are useful to prevent, alleviate or mitigate exercise-induced acidosis and/or acidemia. There is provided a method to prevent, alleviate or mitigate exercise-induced acidosis and/or acidemia in a subject, comprising the step of ingesting the nutritional supplement composition comprising the ingestible particles.

According to one further aspect, the ingestible particles are useful in the nutritional supplement composition to prevent, alleviate or mitigate exercise-induced acidosis and/or acidemia.

According to a further aspect, the composition comprising the ingestible particles and the kit are furthermore useful to treat metabolic acidosis and/or acidemia. This implies that the composition is a pharmaceutical composition. There is provided a method of treating metabolic acidosis and/or acidemia in a human subject in need thereof, comprising the step of ingesting a pharmaceutically effective amount of the composition comprising the ingestible particles. The composition and the kit are useful in a method of treating metabolic acidosis and/or acidemia in a human subject in need thereof, comprising the step of ingesting a pharmaceutically effective amount of the composition comprising the ingestible particles.

According to a further aspect, the ingestible particles are furthermore useful to treat metabolic acidosis and/or acidemia. There is provided a method of treating metabolic acidosis and/or acidemia in a human subject in need thereof, comprising the step of ingesting a pharmaceutically effective amount of the ingestible particles. The ingestible particles are useful in a method of treating metabolic acidosis and/or acidemia in a human subject in need thereof, comprising the step of ingesting a pharmaceutically effective amount of the ingestible particles.

Preferred embodiments and further aspects are defined in the appended claims, listed embodiments and throughout the application text.

### BRIEF DESCRIPTION OF THE DRAWINGS

FIG. 1 shows plasma standard bicarbonate concentrations measured before and after ingestion of sodium bicarbonate particles.

FIG. 2 shows plasma standard bicarbonate concentrations measured before and after ingestion of sodium bicarbonate particles.

FIG. 3 shows uptake of bicarbonate from the sodium bicarbonate particles in combination with either a high-viscosity vehicle or a vehicle of lower viscosity.

FIG. 4 shows blood plasma bicarbonate levels measured after intake of sodium bicarbonate particles.

FIG. 5 compares progress of the dissolution of sodium bicarbonate in the form of granulate, 0.5-1.0 mm, with the form of minitablets, 1.5×3.0 mm.

FIG. 6 shows release of NaHCO₃ from minitablets over time when dispersed in different vehicles and under different conditions.

FIG. 7 compares viscosity and shear-thinning properties of two vehicles.

FIG. 8 shows an oscillatory test for a viscoelastic medium.

FIG. 9 shows effects of blood pH and plasma bicarbonate concentration for an athlete ingesting the inventive nutritional supplement composition.

### DETAILED DESCRIPTION

The nutritional supplement composition provided herein is a suspension comprising ingestible particles dispersed in a viscous aqueous medium.

The ingestible particles comprise sodium bicarbonate and have a well-defined size. The ingestible particles are particles are useful per se in nutritional supplement compositions to improve high-intensity exercise performance. Intake of NaHCO₃ particles according to the invention can be demonstrated to reduce the gastrointestinal side effects seen following intake of a comparable amount of NaHCO₃ in solution or in capsules.

Needless to say, the ingestible particles are designed for releasing the sodium bicarbonate in the body, and more specifically for releasing the sodium bicarbonate in the small intestine. The ingestible particles are designed for releasing the sodium bicarbonate prior to the large intestine.

The ingestible particle is comprising sodium bicarbonate salt, wherein the particle has a size in the range of more than 1.0 mm and not more than 5.0 mm, such as not more than 4.0 mm, with a thickness in at least one dimension of more than 1.0 mm but not more than 2.5 mm, such as not more than 2.0 mm, such as not more than 1.8 mm; and wherein the particle contains more than 50% (w/w) of the sodium bicarbonate. The ingestible particle typically has a size in the range of 1.2-5.0 mm, with a thickness in at least one dimension of 1.2-2.0 mm.

Being smaller than 2.0 mm in at least one dimension provides a good palatability and at the same time allows the ingestible particles to freely pass the pyloric sphincter and therefore has a short gastric transit time which is beneficial. This results in a low level of dissolution of NaHCO₃ in the gastric chamber, significantly reducing potential upper GI distress. The terms “dissolution” and “release” are used interchangeably in this document.

The ingestible particles do not disintegrate; instead the sodium bicarbonate dissolves or erodes successively from the particle surface over time. This is firstly reducing potential upper GI distress, but secondly—equally important—provides a prolonged duration of the desired increase in blood [HCO₃⁻] and pH.

The mechanism for uptake of sodium bicarbonate in the intestine goes via the active uptake of sodium, which involves Na⁺/H⁺ exchange. The protons thus secreted into the intestine will neutralize the bicarbonate which forms carbonic acid which, in turn, forms water and CO₂. The last step is catalyzed by carboanhydrase in the luminal wall of the small intestine. The dissolved CO₂ will readily diffuse through cell membranes and enter the blood stream. There is no scientific evidence for transport of bicarbonate ions as such over cell membranes being of any importance.

The net effect of these processes will be an increase of both sodium and bicarbonate (cations and anions respectively) and pH in extracellular liquid (blood and interstitial liquid).

By time, bicarbonate will be distributed further to other compartments in the body. Bicarbonate will be eliminated by renal secretion so that most of the alkalizing effect of bicarbonate will have disappeared within 24-36 h.

Neutralization of bicarbonate in the stomach and in the small intestine are equivalent in terms of alkalizing effect on the human body. While excessive exposure to bicarbonate in the stomach should be avoided, some degree of exposure is believed to be possible while still achieving high GI tolerability.

Partial, but limited, release of sodium bicarbonate will occur in the stomach. Part of the sodium bicarbonate thus dissolved will react with gastric acid to produce carbonic acid/carbon dioxide, while the main part of it will be transported into the small intestine.

Preferably, substantially all sodium bicarbonate in the particles is fully dissolved and available for uptake into the blood before leaving the small intestine. This prevents release of sodium bicarbonate in the large intestine and reduces potential lower GI distress.

Accordingly, the ingestible particles can be used as a nutritional supplement to improve high-intensity exercise performance, without the risk of severe GI distress as observed with other types NaHCO₃ formulations.

It is preferred that the ingestible particle is comprising more than 65% (w/w) of the sodium bicarbonate, such as more than 75% (w/w), such as more than or equal to 80% (w/w) of the sodium bicarbonate.

It is also preferred that the ingestible particle is comprising less than 90% (w/w) of the sodium bicarbonate, such as less than or equal to 85% (w/w) of the sodium bicarbonate.

Preferably, each ingestible particle comprises less than 80 mg sodium bicarbonate, such as less than 50 mg, preferably less than 30 mg, more preferably less than 20 mg sodium bicarbonate. Further preferably, each ingestible particle comprises more than 5 mg sodium bicarbonate, such as more than 10 mg, preferably more than 15 mg sodium bicarbonate. Typically, each tablet is comprising 5-30 mg, e.g. 10-20 mg, such as 15-20 mg sodium bicarbonate.

The ingestible particle has a thickness in at least one dimension of ≤2.5 mm, such as ≤2.0 mm. It is demonstrated herein that athletes consider that particles having a thickness of less than or equal to 2.0 mm, e.g. 1.5 mm, are considerably easier to swallow than particles with a thickness larger than 2.0 mm, e.g. 2.3 mm. The palatability may be further improved by combining the particles with a viscous fluid which is ingested. It is furthermore considered that a thickness of less than or equal to 2.5 mm, such as less than or equal to 2.0 mm allows the particles to rather freely leave the stomach through the pyloric sphincter, and therefore decreases the risk for GI distress in the stomach.

The ingestible particle preferably has a thickness in at least one dimension of ≤1.8 mm, such as ≤1.6 mm, preferably ≤1.5 mm. These dimensions ensure a good palatability and at the same time allows the ingestible particles to freely pass the pyloric sphincter and therefore has a short gastric transit time which is beneficial. This results in a low level of dissolution of NaHCO₃ in the gastric chamber, significantly reducing potential GI distress.

The ingestible particle could have a thickness in at least one dimension of >1.0 mm, preferably ≥1.2 mm, e.g. ≥1.5 mm. It has been demonstrated that thinner particles (≤0.5 mm) are associated with a faster dissolution time in the gastric chamber, which may in turn cause GI distress. It is also demonstrated herein that particles having a size of 0.5-1.0 mm are associated with moderate GI symptoms. The lower limit for the thickness ensures that the extent of dissolution in the mouth is minimal and the rate of release in the stomach is adequately attenuated, without any need for an enteric coating. The ingestible particle, e.g. the tablets or mini tablet, is preferably non-layered. This implies that the particle is essentially homogenous and that it does not contain any coating. The particle is thus not multi-layered. Preferably, the ingestible particle is a non-layered, uncoated tablet. This is useful to ensure efficient release of the sodium bicarbonate salt in the small intestine, which means that the bicarbonate content is available for uptake into the blood. Typically, the sodium bicarbonate content in the ingestible particle is fully dissolved within less than 3 h after ingestion, preferably less than 2 h after ingestion. A slower dissolution, e.g. more than 4 hours, leads to release of sodium bicarbonate in the large intestine, which in turn is associated with undesirable lower GI symptoms, such as diarrhea.

The ingestible particle preferably has a size of ≤4.0 mm, such as ≤3.5 mm, such as ≤3.0 mm, such as ≤2.0 mm, or even ≤1.8 mm or ≤1.6 mm. Suitable particle sizes are e.g. 1.2-4.0 mm, e.g. 1.2-3.0 mm, e.g. 1.2-2.0 mm or 1.2-1.8 mm. These dimensions ensure a good palatability with a sufficient nutritional intake of bicarbonates. Larger particle sizes than 5.0 mm, such as large capsules, are more difficult to ingest, especially for a performing athlete.

It is preferred that the ingestible particle is spheroid shaped. Typically, the ingestible particle can have a curvature where the side height is less than the overall thickness. Preferred formats for the ingestible particles include pellets, beads, tablets and granules. A particularly preferred format is a tablet, or a mini tablet.

In certain embodiments, the somewhat spheroid particles have a diameter/thickness ratio of 3:2 to 3:1, or 1:3 to 2:3.

In one embodiment, the ingestible particle is a tablet or a mini tablet with a thickness of more than 1.0 mm but not more than 2.0 mm and a diameter of more than 1.0 mm but not more than 5.0 mm, such as a thickness of 1.2-2.0 mm and a diameter of 1.2-5.0 mm. The tablet preferably has a thickness of ≤1.8 mm, such as ≤1.6 mm, preferably ≤1.5 mm. These dimensions ensure a good palatability and at the same time allows the tablets to freely pass the pyloric sphincter and therefore has a short gastric transit time which is beneficial. This results in a low level of dissolution of NaHCO₃ in the gastric chamber, significantly reducing potential GI distress. The tablet could have a thickness of >1.0 mm, most preferably >1.2 mm. It is demonstrated herein that thinner particles (s 0.5 mm) are associated with a faster dissolution time in the gastric chamber, which may in turn cause GI distress. It is also demonstrated herein that particles having a size of 0.5-1.0 mm are associated with moderate GI symptoms. The tablet preferably has a diameter of ≤4.0 mm, such as ≤3.5 mm, such as ≤3.0 mm, such as ≤2.0 mm, or even ≤1.8 mm or ≤1.6 mm. Suitable tablet diameters are e.g. 1.2-4.0 mm, e.g. 1.2-3.0 mm, e.g. 1.2-2.0 mm or 1.2-1.8 mm. These dimensions ensure a good palatability with a sufficient nutritional intake of sodium bicarbonate. Larger tablet sizes than 5.0 mm are more difficult to ingest, especially for a performing athlete.

It is preferred that the particles, e.g. the tablets or mini tablets, are non-disintegrating. Since the ingestible particles do not disintegrate, the sodium bicarbonate dissolves or erodes successively from the particle surface over time. This reduces potential GI distress and provides a prolonged duration of the desired increase in blood [HCO₃-] and pH.

The ingestible particle is not dissolved to any large degree in the mouth or in the stomach. This reduces potential GI distress. Since the particle is present for a considerably longer time in the stomach, more sodium bicarbonate is released in the stomach than in the mouth. A suitably delayed release of sodium bicarbonate from the ingestible particle can advantageously be achieved by incorporating the ingestible particles in a viscous vehicle, such as a gel or other viscoelastic medium.

The sodium bicarbonate content in the ingestible particle is fully dissolved before leaving the small intestine, which means that the sodium bicarbonate content is available for uptake into the blood. Typically, the sodium bicarbonate content in the ingestible particle is fully dissolved within less than 3 h after ingestion, preferably less than 2 h after ingestion. As demonstrated in the examples, this is advantageous compared to e.g. larger capsules as it ensures that the sodium bicarbonate is available for uptake into the blood. Preferably, the time to reach 90% (T90% max) of maximum blood plasma bicarbonate concentration after ingestion of the ingestible particle is less than 3 h after ingestion. A slower dissolution and associated higher T90% max, e.g. more than 4 hours, leads to release of sodium bicarbonate in the large intestine, which in turn is associated with undesirable lower GI symptoms, such as diarrhea. It is preferred that the ingestible particle does not have any enteric coating as this delays the dissolution and associated T90% max. It is thus preferred that the ingestible particle does not have any coating.

In addition to the sodium bicarbonate salt, the ingestible particle may comprise excipients, summing up to 100% (w/w) together with the sodium bicarbonate salt. The ingestible particle may thus further comprise at least one of a binder, a lubricant and a glidant. The ingestible particles may also comprise further excipients.

In certain embodiments, the ingestible particle is further comprising a binder, typically in an amount of 1-50% (w/w), e.g. 1-30% (w/w) or 1-15% (w/w). The binder component is selected to fulfil the requirement on gradual release of sodium bicarbonate by erosion of the particle as described above. A preferred binder is selected from polyvinylpyrrolidone (PVP), calcium carbonate, calcium phosphate, hydroxypropyl cellulose (HPC) and polysaccharides, and combinations thereof. Further preferred binders are microcrystalline cellulose and gelatin which can also in the combinations as set out above. Preferred polysaccharide binders are selected from high molecular weight alginates, pectins, gum tragacanth and gum acacia, and combinations thereof, such as high molecular weight alginates, pectins and gum acacia, and combinations thereof. In certain embodiments, the binder is selected from calcium carbonate and gum acacia, and combinations thereof. In one preferred embodiment, the binder contains HPC. HPC provides a good mechanical stability to the particle. In certain preferred embodiments, the binder is HPC; such as in an amount of 1-30% (w/w), 1-15% (w/w) or 5-15% (w/w) of the ingestible particle. In certain embodiments, the binder is selected from HPC and gum acacia, and combinations thereof.

In certain embodiments, the ingestible particle is further comprising a lubricant, typically in an amount of 0.1-10% (w/w), e.g. 1-5% (w/w). A preferred lubricant is selected from stearic acid, magnesium stearate, sodium stearyl fumarate, and combinations thereof. In certain embodiments, the lubricant is magnesium stearate.

In certain embodiments, the ingestible particle is further comprising a glidant, typically in an amount of 0.1-5% (w/w), e.g. 1-3% (w/w). A preferred glidant is fumed silica (anhydrous colloidal silica).

In certain embodiments, the ingestible particle is further comprising further excipients selected from sugars and complex carbohydrates, typically in an amount of 1-50% (w/w), e.g. 1-40% (w/w), e.g. 1-30% (w/w) e.g. 1-20% (w/w), e.g. 1-10% (w/w). Preferred sugars are selected from glucose, fructose, sucrose and isomaltulose, typically in an amount of 1-50% (w/w), e.g. 1-40% (w/w), e.g. 1-30% (w/w), e.g. 1-20% (w/w), e.g. 1-10% (w/w). Preferred complex carbohydrates are selected from maltodextrin, dried glucose syrup and dried fructose syrup, typically in an amount of 1-50% (w/w), e.g. 1-40% (w/w), e.g. 1-30% (w/w), e.g. 1-20% (w/w), e.g. 1-10% (w/w).

The ingestible particles could preferably have one of the following compositions (summing up to 100 wt %):
(A) Sodium bicarbonate 75-85 wt %; Calcium carbonate 5-10 wt %; Hydroxypropyl cellulose 5-10 wt %; Maltodextrin 0.1-1.0 wt %; Gum acacia 1-3 wt %; Magnesium stearate 1-3 wt %; and Anhydrous colloidal silica 0.1-1.0 wt %.
(B) Sodium bicarbonate 82-92 wt %; Hydroxypropyl cellulose 5-15 wt %; Magnesium stearate 1-3 wt %; and Anhydrous colloidal silica 0.1-1.0%.
(C) Sodium bicarbonate 80-90 wt %; Calcium phosphate 5-15 wt %; Gum acacia 1-5 wt %; Magnesium stearate 1-3 wt %; and Anhydrous colloidal silica 0.1-1.0 wt %.

Preferably, the compositions are formulated as minitablets with a diameter within the range of 1.2-5.0 mm and a thickness within the range of 1.2-2.0 mm.

The nutritional supplement composition is a suspension comprising the ingestible particles dispersed in an aqueous medium. The ingestible particles are preferably suspended in the aqueous medium to facilitate intake and to improve palatability. The aqueous medium may e.g. be a viscous solution or a colloidal suspension.

The ingestible particles shall preferably be freshly dispersed in the aqueous medium shortly before ingestion so as to prevent undesired dissolution of the sodium bicarbonate prior to ingestion. The resulting suspension shall preferably be ingested within 15 minutes after preparation. The released amount of sodium bicarbonate shall be as low as possible prior to ingestion, preferably less than 20% of the total amount ingested, such as less than 15% of the total amount ingested.

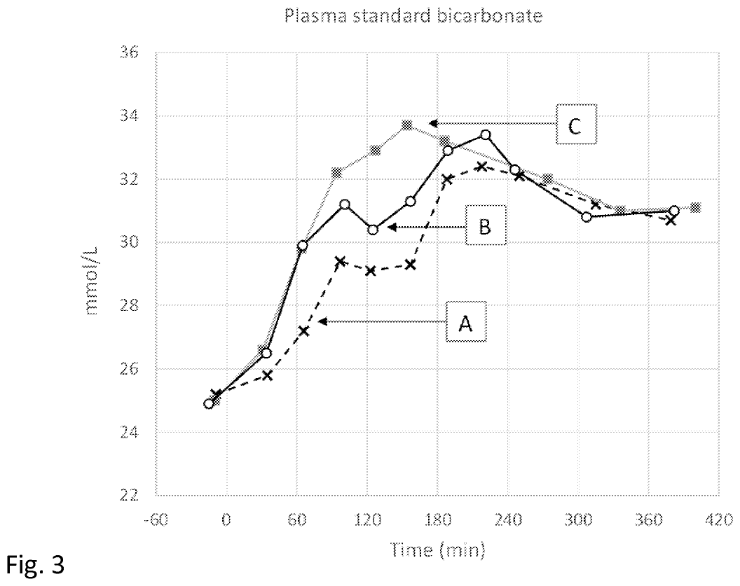

It is preferred that the aqueous medium is a viscous aqueous medium, such as a viscous aqueous liquid, such as a viscous aqueous solution, to facilitate intake and to improve palatability. Preferably, the viscous aqueous medium is a viscoelastic medium. A preferred viscoelastic medium is a gel. The viscous aqueous medium may also be a viscous liquid. Since sodium bicarbonate has a high density, the ingestible particles containing high amounts, up to 85% or even higher, thereof will also have a high density. If these particles are dispersed in water, they will rapidly sink to the bottom of the vessel. The viscosity of the medium should be high enough to prevent sedimentation of the ingestible particles when dispersed in the viscous medium until the dispersion has been ingested. It is preferred that the sedimentation rate of a single ingestible particle in the unstirred viscous aqueous medium is less than 70 mm/min, such as less than 20 mm/min, such as less than 10 mm/min. The sedimentation rate of minitablets in a medium of intermediate viscosity, as in Example 2, is approximately 60 mm/min, c.f. Example 8. It is particularly preferred that the sedimentation rate of a single ingestible particle in the unstirred viscous aqueous medium is less than 5 mm/min, preferably less than 2 mm/min, more preferably less than 1 mm/min. The sedimentation rate of minitablets in a viscous medium, as in Example 1, is less than 0.5 mm/min, c.f. Example 8. It is also demonstrated herein (FIG. 3) that using a vehicle with high viscosity achieves a faster uptake of sodium bicarbonate during the first 90 min after intake, possibly due to faster gastric emptying of the particles. In addition, a viscous vehicle improves the palatability of the composition and ensures that the particles are readily ingested and do not remain in the vessel. A further advantage of a viscous vehicle is that it slows down mixing of the vehicle, e.g. when ingested, and thereby decreases the rate of erosion for the tablets in the nutritional supplement composition.

Without wishing to be limited to any particular theory, it is envisaged that the viscous medium protects the particles from direct exposure to the highly acidic environment of the stomach wall, where hydrochloric acid is excreted. It is considered that this may protect the integrity of the particles and is believed to prevent or decrease gastrointestinal problems which could otherwise occur.

The interactive combination of the ingestible sodium bicarbonate particles and the viscous aqueous medium in the present nutritional supplement composition prevents acidosis, including exercise-induced acidosis, in a surprisingly efficient manner: The size of the ingestible particles allows both for a facilitated intake with improved palatability and a significant reduction in GI side effects. The viscous aqueous medium not only facilitates intake of sufficient amounts of sodium bicarbonate by keeping the particles dispersed in the medium, but also delays the release of sodium bicarbonate from the particles, both prior to ingestion and in the stomach. The viscous aqueous medium is considered to delay release of the sodium bicarbonate from the ingested minitablets until the composition has reached the small intestine, where it is efficiently absorbed.

In one advantageous embodiment of the nutritional supplement composition, the viscous aqueous medium is a viscoelastic medium, preferably, in the form of a gel. Minitablets are dispersed in the viscoelastic medium prior to ingestion of the combined products. Preferably, the viscoelastic medium is a gel, such as an aqueous semisolid gel with viscoelastic properties. The viscoelastic medium prevents sedimentation and facilitates ingestion of the particles. A further advantage of a viscoelastic vehicle is that it slows down mixing of the medium, e.g. when ingested, and thereby decreases the rate of erosion for the tablets in the nutritional supplement composition. As demonstrated in Example 7, the viscoelastic vehicle is considered as useful to substantially delay release of the NaHCO₃ from the minitablets until the composition has been ingested.

One characteristic of a viscoleastic medium is that it has shear-thinning properties or pseudoplastic properties. Compared to a medium which is viscous but not viscoelastic, a viscoelastic medium is viscous when at rest, but less viscous when flowing at speed or when agitated, such as when being swallowed.

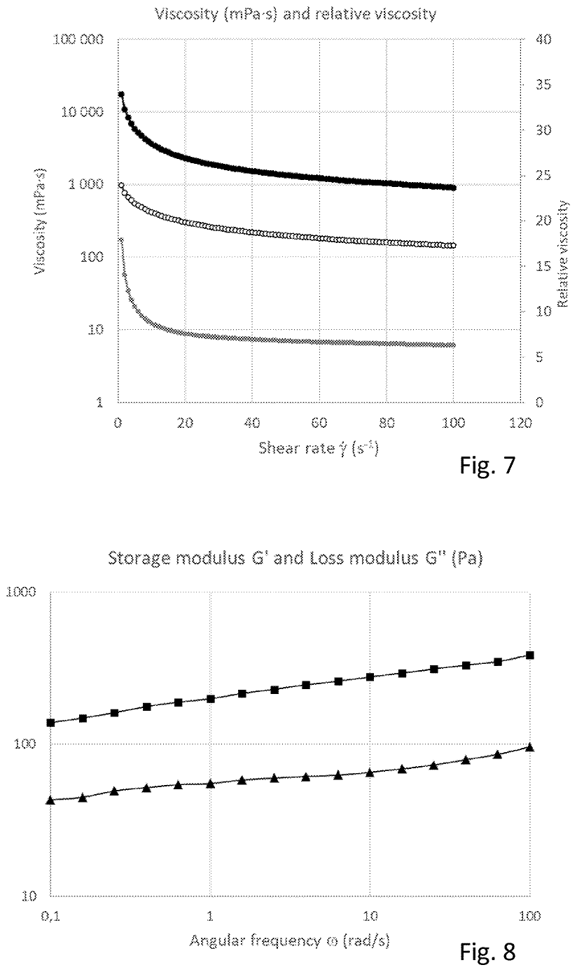

Referring to Example 9 and FIG. 7, viscosity can be e.g. determined at 20 °C using a shear rate controlled rheometer (Model 302, Anton Paar, Germany), using a parallel plate geometry (plate diameter 50 mm, gap 100 μm). In this setup, the viscosity of water is approximately constant at 1 mPa·s, at shear rates between 0-100 s⁻¹. For avoidance of doubt, viscous aqueous media as defined herein are considerably more viscous than water. Viscous aqueous media as defined herein typically exhibit a viscosity of at least 50 mPa·s, such as at least 100 mPa·s at shear rates between 0-100 s⁻¹, such as at 40 s⁻¹. Preferably, aqueous media as defined herein exhibit a viscosity of at least 300 mPa·s, such as at least 500 mPa·s at shear rates between 0-100 s⁻¹, such as at 40 s⁻¹. In addition to the viscous properties, viscoelastic media as defined herein typically exhibit a significantly higher viscosity at shear rates between 0-10 s⁻¹ than at shear rates between 20-100 s⁻¹. Viscoelastic media as defined herein typically exhibit a typically exhibit a viscosity at a shear rate of 5 s⁻¹ that is at least twice the viscosity than at a shear rate of 40 s⁻¹. The specific values above are relevant for this specific setup, but the skilled person can with reference to Example 9 easily determine corresponding values for viscosity and viscoelasticity in other experimental setups. For avoidance of doubt, the viscosity values determined refer to the viscous aqueous medium prior to combination with the ingestible particles, when this property can be determined with this experimental setup.

The viscoelastic medium (vehicle) is advantageous as it slows down mixing of the medium, e.g. when ingested, and thereby decreases the rate of erosion for the tablets in the nutritional supplement composition. In contact with water, bicarbonate will be released from the minitablets in a controlled manner. When minitablets are surrounded by the viscoelastic medium, the release will be slower than in a low-viscosity liquid, due to water close to the tablets being stagnant. In the stomach, the viscoelastic medium also will to some extent act as a barrier against the gastric juice.

In general, a gel can be described as a colloid in which the disperse phase has combined with the dispersion medium to produce a semisolid material with both viscous and elastic properties, where the elastic property dominates. Viscoelastic properties can be described by oscillatory tests, where phase shift between strain (γ, dimensionless) and stress (τ, N/m²) is measured. For phase shifts between 45° and 90°, fluid properties are dominating, while for phase shifts between 0° and 45°, elastic properties, typical for a gel, are dominating. The complex shear modulus can be described as a vector G having the perpendicular components G′ (storage of elastic energy, storage modulus) and G″ (loss of energy by viscous dissipation, loss modulus). Thus, a gel is characterized by G′ being larger than G″. A system is gel-like at a given frequency as soon as G′ (storage modulus which concerns the solid-like response part of the material) is higher than G″ (loss modulus which concerns the liquid-like response on the material).

Gelled products are characterized by having a relatively soft and chewy texture. Typical gelled products include gelatine based products as well as products based on certain types of carrageenan, alginate, starches, agarose, β-glucan, gellan gum, pectin or cellulose compounds. In general, a gel can be described as a colloid in which a dispersed phase has combined with the dispersion medium to produce a semisolid material, e.g., a jelly.

In a gel, the gel structure will prevent tablets from sedimentation/sinking despite their high density. It also contributes to an improved perceived palatability for the nutritional supplement composition. The gel will thereby also facilitate ingestion of the minitablets.

For avoidance of doubt, the ingestible particles comprising sodium bicarbonate are not constituting the dispersed phase of the gel system. The ingestible particles comprising sodium bicarbonate are themselves dispersed in the gel. The gel, in turn, can be described as a colloid in which a dispersed phase (which is not the ingestible particles comprising sodium bicarbonate) has combined with the dispersion medium to produce a semisolid material.

In certain embodiments, the viscous aqueous medium comprises one or more natural polymers dissolved in water as a thickener. Preferred natural polymers are selected from polysaccharides, such as native and modified starch, xanthan gum, guar gum, karragenan, alginate, pectin, and combinations thereof. Modified starch provides viscoelastic properties to the medium with good stability. A preferred modified starch is acetylated distarch adipate, preferably in an amount of more than 2 wt %, such as more than 3 wt % of the viscous aqueous medium to obtain highly useful viscoleastic properties. For high palatability, a combination of starch and a smaller amount of a more potent thickener, such as xanthan gum is advantageous. Preferred natural polymers are a combination of
(a) native or modified starch; and
(b) xanthan gum or guar gum;
in a relative weight ratio (a):(b) of from 99:1 to 90:10.

In a preferred embodiment, the viscous aqueous medium comprises starch or modified starch, preferably modified starch, as the natural polymers. These will be attacked by amylases, leading to weakening the gel, which will facilitate the release of sodium bicarbonate from the minitablets in the small intestine. Bicarbonate release is facilitated and expected to be completed before minitablets reach the colon.

In some embodiments, the viscous aqueous medium comprises one or more sugars or complex carbohydrates dissolved in water. Preferred sugars are selected from glucose, fructose, sucrose and isomaltulose. Preferred complex carbohydrates are selected from starch, maltodextrin, glucose syrup and fructose syrup.

The viscous aqueous medium could preferably have one of the following compositions (balanced with water to 100 wt %):
(A) 4-12 wt % maltodextrin; 3-10 wt % fructose; 2-7 wt % acetylated distarch adipate; and 0.1-1.0 wt % xanthan gum.
(B) 6-14 wt % maltodextrin, 2-5 wt % fructose, 1-3 wt % acetylated distarch adipate and 0.1-1.0 wt % xanthan gum.

In some embodiments of the nutritional supplement composition, the total amount of sodium bicarbonate salt in the ingestible particles in one serving is more than 10 g, preferably more than 15 g. In some embodiments of the nutritional supplement composition, the total amount of sodium bicarbonate salt in the ingestible particles in one serving is less than 50 g, preferably less than 40 g, more preferably less than 30 g.

In one embodiment, the nutritional supplement composition is a suspension comprising the ingestible particles dispersed in an aqueous medium. The particles have a size in the range of 1.2-5.0 mm, with a thickness in at least one dimension of 1.2-2.0 mm. The particles contain more than 65% (w/w) of the sodium bicarbonate salt. The ingestible particles are suspended in a viscous medium, e.g. containing maltodextrin and a sugar, such as fructose, dissolved in water, to facilitate intake and to improve palatability.

There is also provided a kit for preparing a nutritional supplement composition, the kit comprising ingestible particles as defined herein; and an aqueous medium as defined herein. The kit is useful for preparing the nutritional supplement composition as a suspension comprising the ingestible particles dispersed in the aqueous medium. The resulting nutritional supplement composition is useful to improve high-intensity exercise performance. In some embodiments, the total amount of sodium bicarbonate salt in the ingestible particles in one serving is more than 10 g, preferably more than 15 g. In some embodiments of the nutritional supplement composition, the total amount of sodium bicarbonate salt in the ingestible particles in one serving is less than 50 g, preferably less than 40 g, more preferably less than 30 g.

According to a further aspect, the ingestible particles are useful in a nutritional supplement composition to improve high-intensity exercise performance. Specifically, the ingestible particles are useful in the nutritional supplement composition disclosed herein to improve high-intensity exercise performance.

It is furthermore realized that the nutritional supplement composition comprising the ingestible particles and the kit are useful to prevent, alleviate or mitigate exercise-induced acidosis and/or acidemia. The ingestible particles are as such useful to prevent, alleviate or mitigate exercise-induced acidosis and/or acidemia. Supplementation with sodium bicarbonate can also be useful to prevent, alleviate or mitigate exercise-induced acidosis, i.e. an imbalance in the body's acid-base balance induced by exercise. Exercise-induced acidosis can lead to acidemia, which is defined as arterial blood pH that is lower than 7.35. Supplementation with sodium bicarbonate can also be useful to prevent, alleviate or mitigate exercise-induced acidemia.

According to a further aspect, there is provided a method for improving high-intensity exercise performance, comprising the step of ingesting the nutritional supplement composition comprising the ingestible particles. Specifically, the nutritional supplement composition is as defined herein. Preferably, the nutritional supplement composition is freshly prepared by mixing the ingestible particles and the aqueous medium shortly before ingestion, e.g. less than 5 minutes before ingestion. This ensures that the particles are essentially intact and non-dissolved when ingested.

There is provided a method to prevent, alleviate or mitigate exercise-induced acidosis and/or acidemia in a subject, comprising the step of ingesting the nutritional supplement composition comprising the ingestible particles. Typically, ingestible particles containing a total amount of 0.10-0.40 g bicarbonate salt/kg body mass are thereby ingested; preferably 0.20-0.35 g bicarbonate salt/kg body mass; preferably 0.25-0.30 g bicarbonate salt/kg body mass.

In a preferred use or method as defined herein, ingestible particles containing a total amount of 0.10-0.40 g sodium bicarbonate salt/kg body mass are thereby ingested; preferably 0.20-0.35 g sodium bicarbonate salt/kg body mass; preferably 0.25-0.30 g sodium bicarbonate salt/kg body mass. It is preferred that a sufficient amount of sodium bicarbonate is ingested so as to achieve an increase of the blood bicarbonate concentration of at least 5 mmol/l, such as at least 6 mmol/l. It is however realized that already an increase of the blood bicarbonate concentration of 1 mmol/, 2 mmol/l, 3 mmol/l or 4 mmol/l can be useful, depending on the situation.

According to a further aspect, the composition comprising the ingestible particles and the kit are furthermore useful to treat metabolic acidosis and/or acidemia, i.e. a medical condition characterized by an imbalance in the body's acid-base balance. This implies that the composition is a pharmaceutical composition. Metabolic acidosis can lead to acidemia, which is defined as arterial blood pH that is lower than 7.35. Clinically, metabolic acidosis may be caused by increased acid production, loss of bicarbonate, or a reduced ability of the kidneys to excrete excess acids.

Acute metabolic acidosis, lasting from minutes to several days, often occurs during serious illnesses or hospitalizations, and is generally caused when the body produces an excess amount of organic acids (ketoacids or lactic acid). A state of chronic metabolic acidosis, lasting several weeks to years, can be the result of impaired kidney function (Chronic Kidney Disease) and/or bicarbonate wasting. The adverse effects of acute versus chronic metabolic acidosis also differ, with acute metabolic acidosis impacting the cardiovascular system in hospital settings, and chronic metabolic acidosis affecting muscles, bones, kidney and cardiovascular health.

There is provided a method of treating metabolic acidosis and/or acidemia in a human subject in need thereof, comprising the step of ingesting a pharmaceutically effective amount of the composition comprising the ingestible particles. The nutritional supplement composition and the kit are useful in a method of treating metabolic acidosis and/or acidemia in a human subject in need thereof, comprising the step of ingesting a pharmaceutically effective amount of the composition comprising the ingestible particles. The metabolic acidosis and/or acidemia may be caused by chronic kidney disease. Typically, ingestible particles containing a total amount of 0.01-0.10 g bicarbonate salt/kg body mass are thereby ingested; preferably 0.02-0.07 g bicarbonate salt/kg body mass; preferably 0.02-0.04 g bicarbonate salt/kg body mass. In preferred embodiments, this corresponds to a daily dose. In some embodiments, the daily amount of sodium bicarbonate salt in the ingestible particles is more than 1 g, preferably more than 2 g. In some embodiments of the composition, the daily amount of sodium bicarbonate salt in the ingestible particles is less than 5 g, preferably less than 4 g, more preferably less than 3 g, such as 2-5 g, 3-5 g or 2-3 g daily. It is desirable that the entire daily dose is taken at a single occasion, and the composition disclosed herein decreases the associated risk of gastrointestinal problems.

The ingestible particles are furthermore useful per se to treat metabolic acidosis and/or acidemia. There is provided a method of treating metabolic acidosis and/or acidemia in a human subject in need thereof, comprising the step of ingesting a pharmaceutically effective amount of the ingestible particles. The ingestible particles are useful in a method of treating metabolic acidosis and/or acidemia in a human subject in need thereof, comprising the step of ingesting a pharmaceutically effective amount of the ingestible particles. The metabolic acidosis and/or acidemia may be caused by chronic kidney disease. Typically, ingestible particles containing a total amount of 0.01-0.10 g bicarbonate salt/kg body mass are thereby ingested; preferably 0.02-0.07 g bicarbonate salt/kg body mass; preferably 0.02-0.04 g bicarbonate salt/kg body mass. In preferred embodiments, this corresponds to a daily dose. In some embodiments, the daily amount of sodium bicarbonate salt in the ingestible particles is more than 1 g, preferably more than 2 g. In some embodiments of the composition, the daily amount of sodium bicarbonate salt in the ingestible particles is less than 5 g, preferably less than 4 g, more preferably less than 3 g, such as 2-5 g, 3-5 g or 2-3 g daily. It is desirable that the entire daily dose is taken at a single occasion, and the ingestible particles disclosed herein decrease the associated risk of gastrointestinal problems.

All publications cited herein are incorporated by reference herein for all purposes.

The present invention will in the following be further illustrated by the following non-limiting examples.

### Examples

### Example 1

Minitablets with a diameter of 3.0 mm, a height of 1.5 mm and a mean weight of 18 mg were produced with the following composition: NaHCO₃85 wt %, Calcium phosphate 10 wt %, gum acacia 2.5 wt %, magnesium stearate 2 wt % and anhydrous colloidal silica 0.5 wt %. Components were mixed in a Turbula mixer. The tablets were compressed using a Fette 52i rotary press, equipped with a force feeder.

Participants had taken a breakfast at least 3 h prior to ingestion of sodium bicarbonate minitablets and vehicle. A viscous vehicle was prepared by blending 22 g maltodextrin, 16 g fructose, 12 g acetylated di-starch adipate and 0.4 g xanthan gum and mixing the blend with 300 g of water. The mixture was ready to use 10 min after mixing. Sodium bicarbonate minitablets corresponding to 0.3 g sodium bicarbonate per kilo bodyweight were added to the mixture and all was ingested within 5 min.

Arterialized capillary blood samples were collected before ingestion and at 30, 60 or 120 min intervals until 8 h after intake. Blood samples were analyzed immediately on a blood gas analyzer.

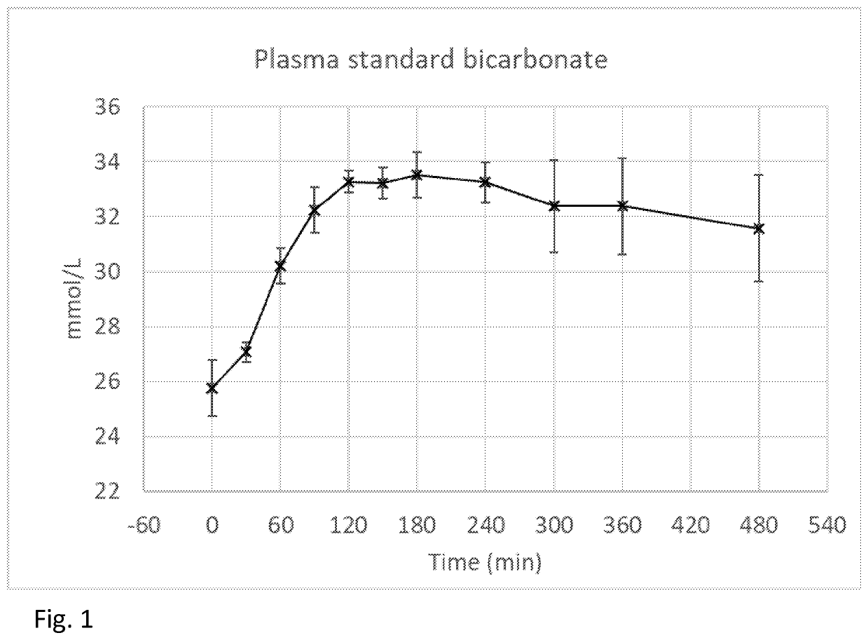

FIG. 1 shows plasma standard bicarbonate concentrations (mean+/−S.D., n=4) measured before and after ingestion of minitablets, corresponding to 0.30 g NaHCO₃ per kilo bodyweight, taken together with 350 g of viscous drink.

At least 90% of the peak bicarbonate concentration was reached at 120 min. The maximum increase from t=0 was around 7 mmol/l.

### Example 2

Minitablets as in Example 1 were used.

The participant had taken a breakfast at least 3 h prior to ingestion of sodium bicarbonate minitablets and vehicle. A viscous vehicle was prepared by blending 33 g maltodextrin, 11 g fructose, 5.4 g acetylated di-starch adipate and 0.6 g xanthan gum and mixing the blend with 300 g of water (intermediate viscosity). Sodium bicarbonate minitablets corresponding to 0.3 g sodium bicarbonate per kilo bodyweight were added to the mixture and all was ingested within 5 min. A light meal was taken at 60 min, consisting of two rice cakes with almond butter and of a drink containing 26 g of maltodextrin and 13 g of fructose dissolved in 300 g water.

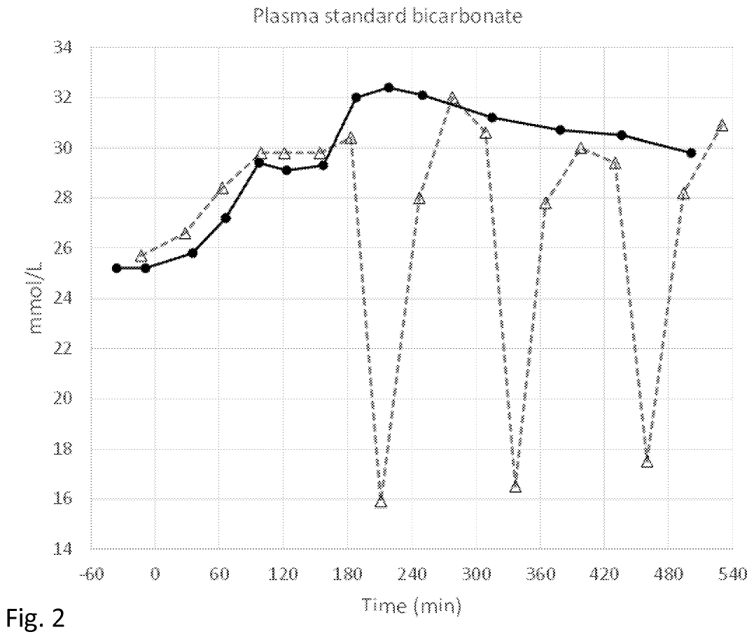

On one of the two experiment days (dashed line in FIG. 2), three 20-min high intensity interval training (HIIT) exercises were conducted, starting at about 3, 5 and 7 h respectively after sodium bicarbonate intake. Arterialized capillary blood samples were collected before and until about 8.5 h after intake.

FIG. 2 shows plasma standard bicarbonate concentrations measured before and after ingestion of minitablets corresponding to 0.30 g NaHCO₃ per kilo bodyweight, taken together with 350 g of the viscous drink (intermediate viscosity).
Solid line: Resting or low intensity exercise (walking) conditions.
Dashed line: Resting and low intensity exercise interrupted by three 20-min high intensity interval training (HIIT) sessions, starting after blood sampling at about 180, 300 and 420 min respectively.

Plasma bicarbonate was lowered by HIIT sessions but was restored before next HIIT session and at the end of the trial, compared to plasma bicarbonate levels resting/low intensity exercise conditions (FIG. 2).

### Example 3

Minitablets as in Example 1 and 2 were used in combination with either a high viscosity vehicle as in Example 1, or a vehicle with intermediate viscosity, as in Example 2. Mixtures containing minitablets corresponding to 0.30 g sodium bicarbonate per kg bodyweight were ingested 2.5-3.5 h after a standard breakfast meal. Standard bicarbonate levels in arterialized capillary blood were monitored shortly before and after intake.

FIG. 3 shows uptake of bicarbonate from minitablets in combination with either a high-viscosity vehicle (solid lines B and C) or a vehicle of intermediate viscosity (dashed line A). In samples A and B, a light meal was taken at 60 min. In sample C, no light meal was taken.

As seen in FIG. 3, standard bicarbonate levels in arterialized capillary blood, monitored shortly before and after intake indicated a faster uptake of bicarbonate during the first 120 min after intake using the high-viscosity vehicle (B and C), possibly due to faster gastric emptying. A light meal taken at 60 min (consisting of two rice cakes with almond butter and of a drink containing 26 g of maltodextrin and 13 g of fructose dissolved in 300 g water) affected uptake of bicarbonate during the following 90-120 min (A and B).

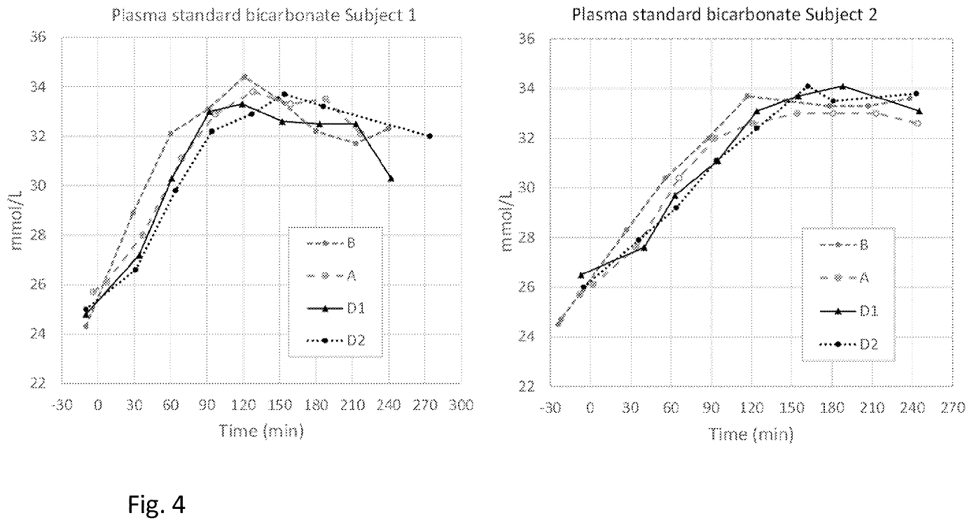

Example 4 Different sodium bicarbonate formulations, each in combination with a vehicle, were ingested by 2-6 subjects on 1-4 occasions. Perceived palatability as well as perceived upper and lower gastrointestinal symptoms 0-6 h after ingestion were recorded. Standard bicarbonate concentrations in capillary blood were monitored before ingestion and up to at least 210 min after ingestion. Results are summarized in Table 1. Examples of bicarbonate levels from single experiments are given in FIG. 4.

FIG. 4 shows blood plasma bicarbonate levels in two different subjects measured after intake of
A: Sodium bicarbonate dissolved in 480 mL of water, containing 33 g of maltodextrin and 17 g of sucrose;
B: Sodium bicarbonate powder (<0.3 mm) mixed with vehicle as in Example 1 directly prior to ingestion;
D1 and D2: Two identical experiments with sodium bicarbonate minitablets and viscous vehicle as in Example 1.

TABLE 1
Perceived palatability and GI symptoms and time to reach 90% of
maximum blood plasma bicarbonate concentration (T90% max) after
ingestion of different bicarbonate and vehicle formulations.

| | | | | | | | | No. of |
|---|---|---|---|---|---|---|---|---|
| | Bicar- | | | | Upper | Lower | T90% | subjects × |
| | bonate | Dimensions | Vehicle | Palat- | GI | GI | max | No. of |
| | source | (mm) | viscosity | ability | symptoms | symptoms | (h) | occasions |
| A | NaHCO₃ | n.a. | Low | 1 | 3 | 2 | 1.5-2.0 | n = 2 × 1 |
| | dissolved | | | | | | | |
| B | NaHCO₃ | <0.3 | High | 2 | 2-3 | 2 | 1.5-2.0 | n = 2 × 1 |
| | powder | | | | | | | |
| C | NaHCO₃ | 0.5-1.0 | High | 3 | 2-3 | 3-4 | 2.5 | n = 2 × 1 |
| | particles | | | | | | | |
| D | 18 mg | 1.5 × 3.0 | High | 4 | 1 | 1 | 2.0-2.5 | n = 6 × 1, |
| | minitablets | | | | | | | 2 × 4 |
| E | 18 mg | 1.5 × 3.0 | Inter- | 3-4 | 1 | 1 | 2.0-2.5 | n = 2 × 4 |
| | minitablets | | mediate | | | | | |
| F | 18 mg | 1.5 × 3.0 | High | 4 | 1 | 1 | n.a. | n = 2 × 4 |
| | placebo | | | | | | | |
| | minitablets | | | | | | | |
| G | 24 mg | 2.3 × 3.0 | High | 3 | 1 | 1 | 1.5-2.5 | n = 2 × 2 |
| | minitablets | | | | | | | |
| H | 27 mg | 2.3 × 3.0 | High | 3 | 1 | 3-4 | ≥4.0 | n = 2 × 1 |
| | enteric | | | | | | | |
| | minitablets | | | | | | | |
| I | 1.2 g | 8 × 25 | Low | 2-3 | 2 | 1-2 | 2.0-2.5 | n = 2 × 2 |
| | capsules | | | | | | | |

A: Sodium bicarbonate dissolved in low viscosity vehicle (sucrose 33 g, maltodextrin 17 g, 480 ml water).
B: Sodium bicarbonate powder added to high viscosity vehicle just before ingestion.
C: Sodium bicarbonate granulate.
D and E: minitablets as in Example 1-3, containing 85% sodium bicarbonate.
F: Placebo minitablets where sodium bicarbonate was replaced by calcium carbonate (50%) and maltodextrin (35%).
G: Minitablets as in Example 1-3, wherein calcium phosphate and gum acacia is replaced by 10 wt % PVP. Sodium bicarbonate content is 87.5 wt %.
H: Minitablets as in G, with an enteric coating of Eudragit ® L30D55.
I: Hypromellose capsules, size 00E, filled with 1.2 g sodium bicarbonate powder (<0.3 mm)
High viscosity vehicle as described in Example 1.
Intermediate viscosity vehicle as described in Example 2.
Palatability: 1 = very low, 5 = very high.
Upper and lower GI symptoms: 1 = no symptoms, 4-5 = severe symptoms.

### Example 5

Minitablets as in Example 1 containing 85% NaHCO₃ were compared with sodium bicarbonate granulate of particle size 0.5-1.0 mm (sample C in Table 1) in terms of in vitro dissolution rate.

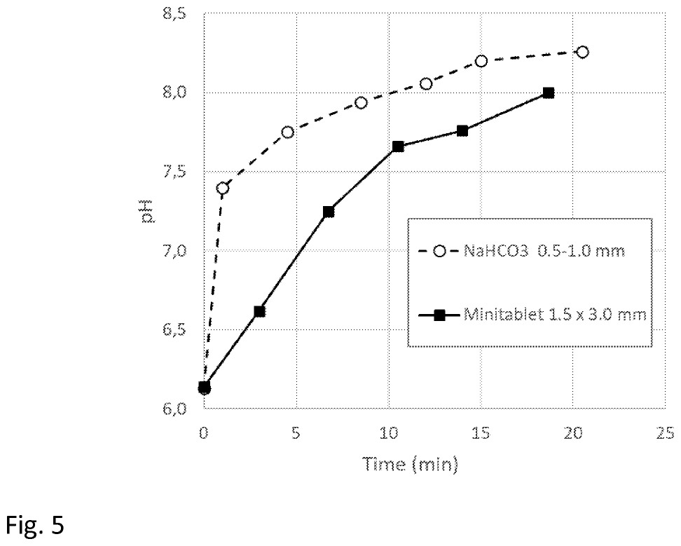

Minitablets or granulate corresponding to 138 mg of sodium bicarbonate were added to 30 ml of simulated intestinal fluid, placed in 50-ml screw capped containers. The containers were intermittently gently shaken during the experiment and pH was measured over time, see FIG. 5.

The rise in pH caused by bicarbonate is substantially delayed with the minitablets compared to the small granules.

### Example 6

(A) Minitablets with a diameter within the range of 1.2-5.0 mm and a thickness within the range of 1.2-2.0 mm were produced with the following composition:
Sodium bicarbonate 80 wt %
Calcium carbonate 8 wt %
Hydroxypropyl cellulose 7 wt %
Maltodextrin 0.5 wt %
Gum acacia 2 wt %
Magnesium stearate 2 wt %
Anhydrous colloidal silica 0.5%.

(B) Minitablets with a diameter within the range of 1.2-5.0 mm and a thickness within the range of 1.2-2.0 mm are produced with the following composition:
Sodium bicarbonate 87.5 wt %
Hydroxypropyl cellulose 10 wt %
Magnesium stearate 2 wt %
Anhydrous colloidal silica 0.5%.

### Example 7

Release of sodium bicarbonate from minitablets in three different experiments.

(A) An equipment similar to the dissolution apparatus of type 2, according to the European Pharmacopoeia (Ph. Eur.) 2.9.3 “Dissolution test for solid dosage forms” was used. Eight minitablets, size 1.5×3.0 mm, with a total weight of 144 mg, containing 126 mg NaHCO₃ and excipients as in Example 6B, were placed on a stainless steel net 40 mm above the bottom of a 500-ml glass beaker, and 25 mm above a magnetic stirring bar rotating. Demineralized water (500 ml) was kept at 37+/−2 °C and stirred at 150 rpm. The concentration of dissolved NaHCO₃, was determined by electric conductivity measurement. As a reference for complete release, conductivity was measured when remains of the tablets could no longer be observed and the conductivity did not increase further within three minutes. More than 75% of the NaHCO₃ was released within 15 min and more than 95% within 30 min from minitablets in demineralized water.

(B) Six containers, each containing eight minitablets as in (A) above were dispersed in 40 g of a viscoelastic medium, a semisolid gel as described in Example 1, and were tumbled at 12 rpm in an incubator kept at 37 °C. Containers were taken out at different time points and after removing and gently washing the minitablets with demineralized water, the gel and wash liquid were mixed and diluted to a total weight of 200 g. The NaHCO₃ concentration was measured by electric conductivity measurement. As a reference for complete release, eight minitablets were kept in 160 ml of demineralized water until totally disintegrated and then mixed with 40 g of gel. When minitablets were kept dispersed in slowly tumbled gel, about 25% of the NaHCO₃ was released within 15 min, about 50% within 30 min and about 75% within 60 min.

(C) Minitablets were dispersed and kept in gel for 15 min at room temperature. The amount NaCO₃ released was determined as in (B). When minitablets were kept dispersed in a gel at room temperature without tumbling, about 10-15% of NaCO₃ was released within 15 min.

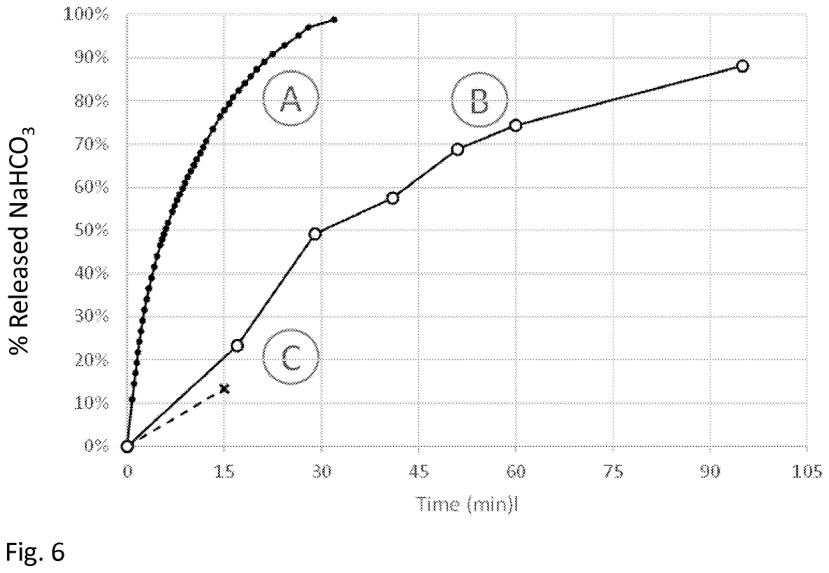

The results are presented in FIG. 6, wherein the released percentage of NaHCO₃ from the minitablets is shown over time for experiments (A)-(C) as detailed above.

It is concluded from these experiments that the release of NaHCO₃ from the minitablets is considerably slower when they are tumbled in a viscoelastic gel vehicle (B) compared to when stirred in a water vehicle (A). In the context of the present invention, the viscoelastic vehicle is considered as useful to delay release of the NaHCO₃ from the ingested minitablets until the composition has reached the small intestine. It is also concluded that when the mini-tablets are dispersed in a gel that is not tumbled (C), the release of NaHCO₃ from the minitablets is considerably slower than when tumbled (B). In the context of the present invention, the viscoelastic vehicle is considered as useful to substantially delay release of the NaHCO₃ from the minitablets until the composition has been ingested. About 10-15% of NaCO₃ was released within 15 min in experiment (C), within which time it is suggested to ingest the product.

### Example 8

Viscoelastic vehicles were tested for sedimentation rate of bicarbonate minitablets as defined herein.

The tablets were of diameter 3 mm, height 1.5 mm, weight 18 mg and density 2.6 mg/mm³. A few tablets were placed beneath the surface of the vehicle and observed for up to 30 min or until having sedimented at least 10 mm.

Vehicle A (A 100%) contained 27 g maltodextrin, 20 g fructose, 15 g acetylated di-starch adipate, 0.5 g xanthan gum, 300 ml water. Dilutions of A with water were also prepared, termed A 80%-A 67%. The vehicle A 67% corresponds to a dilution of A 100% by a factor 1.5. Vehicle E1 is a composition as in Example 1. Vehicle E2 is a composition as in Example 2.

The sedimentation rates for the tested vehicles are presented in Table 2.

TABLE 2

| vehicle | sedimentation rate (mm/min) |
|---|---|
| A 100% | 0 ^{a} |
| A 80% | 0.1 |
| A 75% | 0.2 |
| A 70% | 3.3 |
| A 67% | 30 |
| E1 | 0 (no visible sedimentation within 10 min) |
| E2 | 60 |

### Example 9

(A) Viscosity and shear-thinning properties of the two vehicles A 100% and A 67% from Example 8 were measured using a share rate controlled rheometer (Model 302, Anton Paar, Germany). A parallel plate geometry was used (plate diameter 50 mm, gap 100 μm). Viscosity was measured at 20 °C. The viscosities for the vehicles are presented in FIG. 7 (A-100 and A-67). The relative viscosity A-100/A-67 is also presented in FIG. 7.

As seen in FIG. 7, the viscosity curves of the two hydrocolloid dispersions A-100 and A-67 exhibit shear thinning properties. Viscosities at shear rate 5 s⁻¹: A-100: 5900 mPa·s; A-67 560 mPa·s. Viscosities at shear rate 40 s⁻¹: A-100: 1540 mPa·s; A-67 220 mPa·s.

Viscosity of A-67, obtained by diluting vehicle A-100 by a factor 1.5, resulted in a decrease in viscosity by a factor of about 6 or more, depending on shear rate, as shown in FIG. 7.

(B) An oscillatory test showing elastic component G′ (storage modulus) and viscous component G″ (loss modulus) of complex shear vector G was conducted for Vehicle A (A 100%) from Example 8.

Measurements were made at 20 °C using a shear rate controlled rheometer (Model 302, Anton Paar, Germany). A parallel plate geometry was used (plate diameter 50 mm, gap 100 μm).

The storage modulus G′ and the loss modulus G″ for A 100% are shown in FIG. 8. The ratio G′/G″ is about 3 which means that the sample is a viscoelastic medium and has the properties of a gel.

### Example 10

A well-trained male athlete, body weight 80 kg, performed high-intensity interval training on two consecutive days, following the same exercise protocol on each day. Blood pH and plasma bicarbonate were measured before, during and after high-intensity interval training. After warm-up, 45-s running and 15-s resting was repeated 30 times starting at time 0 with a five min break half-way. Exercise intensity exceeded the anaerobic threshold (plasma lactate exceeding 4 mmol/l). On the second day, a nutritional supplement composition as defined herein was ingested prior to exercise, shortly after the sampling at time −90 min. The nutritional supplement composition contained minitablets as described in Example 7 containing 22 g NaHCO₃, corresponding to 0.275 g/kg body weight, dispersed in a semisolid vehicle as described in Example 1.

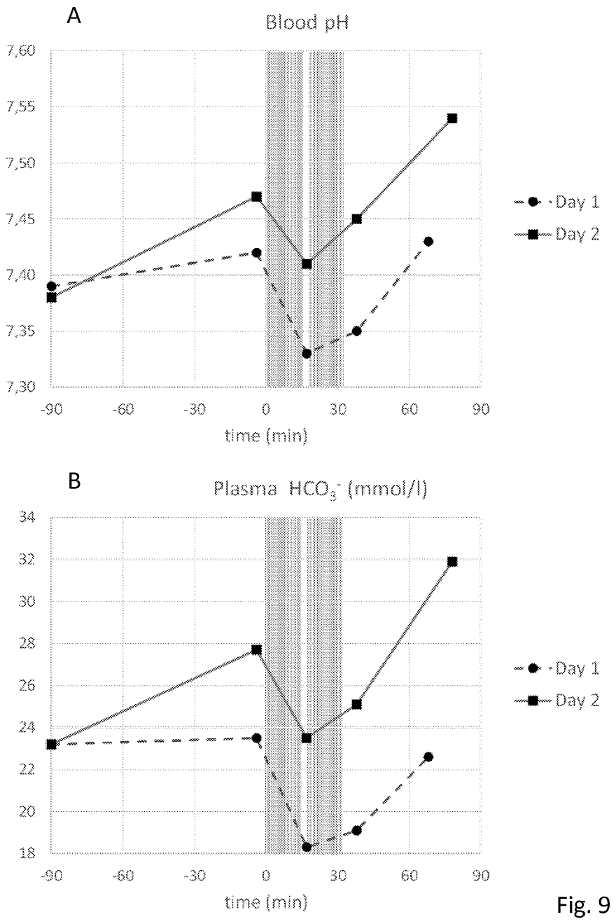

The results are presented in FIG. 9, wherein the upper panel (A) shows blood pH and the lower panel (B) shows plasma bicarbonate concentration. Grey bars illustrate the high-intensity interval training intervals.
Day 1: circles/dotted line; Day 2: squares/solid line.

On the first day (control), exercise resulted in a drop in pH from pH 7.43 to pH 7.35, and a drop in plasma bicarbonate concentration from 24 mmol/l to 18 mmol/l, corresponding to a mild acidosis. On the second day, a pH drop caused by exercise was counteracted by a pH increase caused by ongoing uptake bicarbonate from the ingested nutritional supplement composition. The blood pH remained above 7.40 and the plasma bicarbonate concentration remained above 23 mmol/l. The changes in blood pH and plasma bicarbonate followed the same pattern, as can be seen in FIG. 9

### ITEMIZED LIST OF EMBODIMENTS

1. A nutritional supplement composition which is a suspension comprising ingestible particles comprising sodium bicarbonate, dispersed in an aqueous medium; wherein the particles are tablets with a thickness of 1.0-2.0 mm and a diameter of 1.0-5.0 mm; and wherein the particles contain more than 50% (w/w) of the sodium bicarbonate; and wherein the aqueous medium is a viscous aqueous medium.
2. A nutritional supplement composition according to item 1, wherein the ingestible particles comprise more than 65% (w/w) of the sodium bicarbonate, such as more than 75% (w/w) of the sodium bicarbonate.
3. A nutritional supplement composition according to any one of the preceding items, wherein the ingestible particles comprise less than 90% (w/w) of the sodium bicarbonate, such as less than or equal to 85% (w/w) of the sodium bicarbonate.
4. A nutritional supplement composition according to any one of the preceding items, wherein the ingestible particles are suitable for releasing the sodium bicarbonate in the small intestine.
5. A nutritional supplement composition according to any one of the preceding items, wherein the tablets have a thickness of 1.2-2.0 mm and a diameter of 1.2-5.0 mm.
6. A nutritional supplement composition according to any one of the preceding items, wherein the tablets have a thickness of ≤1.8 mm, such as ≤1.5 mm.
7. A nutritional supplement composition according to any one of the preceding items, wherein the tablets have a diameter of ≤4.0 mm, such as ≤3.0 mm.
8. A nutritional supplement composition according to item 7, wherein the tablets have a diameter of ≤2.0 mm, such as ≤1.8 mm.
9. A nutritional supplement composition according to any one of the preceding items, wherein the ingestible particles are non-disintegrating.
10. A nutritional supplement composition according to any one of the preceding items, wherein the ingestible particles are without any coating, such as without any enteric coating.
11. A nutritional supplement composition according to any one of the preceding items, wherein the ingestible particles are non-layered particles.
12. A nutritional supplement composition according to any one of the preceding items, wherein the ingestible particles are further comprising a binder.
13. A nutritional supplement composition according to item 12, wherein the binder is selected from polyvinylpyrrolidone (PVP), calcium carbonate, calcium phosphate, hydroxypropyl cellulose (HPC), microcrystalline cellulose, gelatin and polysaccharides, and combinations thereof.
14. A nutritional supplement composition according to item 13, wherein the binder is selected from hydroxypropyl cellulose (HPC) and combinations 25 thereof.
15. A nutritional supplement composition according to item 13, wherein the polysaccharides are selected from high molecular weight alginates, pectins, gum tragacanth and gum acacia, and combinations thereof.
16. A nutritional supplement composition according to item 12, wherein the binder is selected from calcium carbonate and gum acacia, and combinations thereof.
17. A nutritional supplement composition according to any one of the preceding items, wherein the ingestible particles are further comprising a lubricant.
18. A nutritional supplement composition according to item 17, wherein the lubricant is selected from stearic acid, magnesium stearate, sodium stearyl fumarate, and combinations thereof; preferably wherein the lubricant is magnesium stearate.
19. A nutritional supplement composition according to any one of the preceding items, wherein the ingestible particles are further comprising a glidant.
20. A nutritional supplement composition according to item 19, wherein the glidant is fumed silica.
21. A nutritional supplement composition according to any one of the preceding items, wherein the ingestible particles are comprising further excipients selected from sugars and complex carbohydrates.
22. A nutritional supplement composition according to item 21, wherein the sugars are selected from glucose, fructose, sucrose and isomaltulose.
23. A nutritional supplement composition according to item 21, wherein the the complex carbohydrates are selected from starch, maltodextrin, dried glucose syrup and dried fructose syrup.
24. A nutritional supplement composition according to any one of items 1 and 4-11, wherein the ingestible particles have the following composition: Sodium bicarbonate 75-85 wt %; Calcium carbonate 5-10 wt %; Hydroxypropyl cellulose 5-10 wt %; Maltodextrin 0.1-1.0 wt %; Gum acacia 1-3 wt %; Magnesium stearate 1-3 wt %; and Anhydrous colloidal silica 0.1-1.0 wt %.
25. A nutritional supplement composition according to any one of items 1 and 4-11, wherein the ingestible particles have the following composition: Sodium bicarbonate 82-92 wt %; Hydroxypropyl cellulose 5-15 wt %; Magnesium stearate 1-3 wt %; and Anhydrous colloidal silica 0.1-1.0%.
26. A nutritional supplement composition according to any one of items 1 and 4-11, wherein the ingestible particles have the following composition: Sodium bicarbonate 80-90 wt %; Calcium phosphate 5-15 wt %; Gum acacia 1-5 wt %; Magnesium stearate 1-3 wt %; and Anhydrous colloidal silica 0.1-1.0 wt %.
27. A nutritional supplement composition according to any of the preceding items, wherein the sedimentation rate of a single ingestible particle in the unstirred viscous aqueous medium is less than 5 mm/min, preferably less than 2 mm/min, more preferably less than 1 mm/min.
28. A nutritional supplement composition according to any one of the preceding items, wherein the viscous aqueous medium is a viscoelastic medium.
29. A nutritional supplement composition according to any one of the preceding items, wherein the viscous aqueous medium is a liquid.
30. A nutritional supplement composition according to item 28, wherein the viscoelastic medium is a gel.
31. A nutritional supplement composition according to any one of the preceding items, wherein the viscous aqueous medium comprises one or more natural polymers dissolved in water as a thickener.
32. A nutritional supplement composition according to item 31, wherein the natural polymers are selected from polysaccharides, such as native and modified starch, xanthan gum, guar gum, karragenan, alginate, pectin, and combinations thereof.
33. A nutritional supplement composition according to item 32, wherein the natural polymers are a combination of
(a) native or modified starch; and
(b) xanthan gum or guar gum;
in a relative weight ratio (a):(b) of from 99:1 to 90:10.
34. A nutritional supplement composition according to item 32, wherein the natural polymers are selected from native and modified starch.
35. A nutritional supplement composition according to item 34, wherein the natural polymers are modified starch.
36. A nutritional supplement composition according to any one of the preceding items, wherein the viscous aqueous medium comprises one or more sugars or complex carbohydrates dissolved in water.
37. A nutritional supplement composition according to item 36 wherein the sugars are selected from glucose, fructose, sucrose and isomaltulose.
38. A nutritional supplement composition according to item 36, wherein the complex carbohydrates are selected from starch, maltodextrin, glucose syrup and fructose syrup.
39. A nutritional supplement composition according to any one of items 1-30, wherein the viscous aqueous medium has the following composition: 4-12 wt % maltodextrin; 3-10 wt % fructose; 2-7 wt % acetylated distarch adipate; and 0.1-1.0 wt % xanthan gum; in water to 100 wt %.
40. A nutritional supplement composition according to any one of items 1-30, wherein the viscous aqueous medium has the following composition: 6-14 wt % maltodextrin, 2-5 wt % fructose, 1-3 wt % acetylated distarch adipate and 0.1-1.0 wt % xanthan gum; in water to 100 wt %.
41. A nutritional supplement composition according to any one of the preceding items, wherein the total amount of sodium bicarbonate in the ingestible particles in one serving is more than 10 g, preferably more than 15 g.
42. A nutritional supplement composition according to item 41, wherein the total amount of sodium bicarbonate in the ingestible particles in one serving is less than 50 g, preferably less than 40 g, more preferably less than 30 g.
43. A kit for preparing a nutritional supplement composition, the kit comprising ingestible particles and an aqueous medium as defined in any one of the preceding items.
44. A kit according to item 43, wherein the nutritional supplement composition is a suspension comprising the ingestible particles dispersed in the aqueous medium.
45. Use of a nutritional supplement composition according to any one of items 1-42 or a kit according to any one of items 43-44 to improve high-intensity exercise performance.
46. Use of a nutritional supplement composition according to any one of items 1-42 or a kit according to any one of items 43-44 to prevent, alleviate or mitigate exercise-induced acidosis and/or acidemia.
47. A method for improving high-intensity exercise performance, comprising the step of ingesting a nutritional supplement composition comprising ingestible particles according to any one of items 1-42.
48. A method according to item 47, wherein ingestible particles containing a total amount of 0.10-0.40 g bicarbonate salt/kg body mass are thereby ingested; preferably 0.20-0.35 g bicarbonate salt/kg body mass; preferably 0.25-0.30 g bicarbonate salt/kg body mass.
49. A method for preventing, alleviating or mitigating exercise-induced acidosis and/or acidemia in a subject, comprising the step of ingesting a nutritional supplement composition according to any one of items 1-42.
50. A method of treating metabolic acidosis and/or acidemia in a human subject in need thereof, comprising the step of ingesting a pharmaceutically effective amount of a composition according to any one of items 1-42.
51. A method according to item 50, wherein the metabolic acidosis and/or acidemia is caused by chronic kidney disease.
52. A method according to any one of items 50-51, wherein ingestible particles containing a total amount of 0.01-0.10 g bicarbonate salt/kg body mass are thereby ingested; preferably 0.02-0.07 g bicarbonate salt/kg body mass; preferably 0.02-0.04 g bicarbonate salt/kg body mass.
53. A composition according to any one of items 1-42 or a kit according to any one of items 43-44 for use as a medicament.
54. A composition according to any one of items 1-42 or a kit according to any one of items 43-44 for use in a method according to any one of items 50-52.
55. Use of a composition according to any one of items 1-42 or a kit according to any one of items 43-44 in the preparation of a medicament for the treatment of metabolic acidosis and/or acidemia.
56. An ingestible particle comprising sodium bicarbonate, wherein the particle is a tablet with a thickness of 1.0-2.0 mm and a diameter of 1.0-5.0 mm, and wherein the particle contains more than 50% (w/w) of the sodium bicarbonate.
57. An ingestible particle according to item 56, comprising more than 65% (w/w) of the sodium bicarbonate, such as more than 75% (w/w) of the sodium bicarbonate.
58. An ingestible particle according to any one of items 56-57, comprising less than 90% (w/w) of the sodium bicarbonate, such as less than or equal to 85% (w/w) of the sodium bicarbonate.
59. An ingestible particle according to any one of items 56-58, wherein the particle is suitable for releasing the sodium bicarbonate in the small intestine.
60. An ingestible particle according to any one of items 56-59, wherein the tablet has a thickness of 1.2-2.0 mm and a diameter of 1.2-5.0 mm.
61. An ingestible particle according to any one of items 56-60, wherein the tablet has a thickness of ≤1.8 mm, such as ≤1.5 mm.
62. An ingestible particle according to any one of items 56-61, wherein the tablet has a diameter of ≤4.0 mm, such as ≤3.0 mm.
63. An ingestible particle according to item 62, wherein the tablet has a diameter of ≤2.0 mm, such as ≤1.8 mm.
64. An ingestible particle according to any one of items 56-63, wherein the particle is non-disintegrating.
65. An ingestible particle according to any one of items 56-64, without any coating, such as without any enteric coating.
66. An ingestible particle according to any one of items 56-65, wherein the ingestible particle is a non-layered particle.
67. An ingestible particle according to any one of items 56-66, further comprising a binder.
68. An ingestible particle according to item 67, wherein the binder is selected from polyvinylpyrrolidone (PVP), calcium carbonate, calcium phosphate, hydroxypropyl cellulose (HPC), microcrystalline cellulose, gelatin and polysaccharides, and combinations thereof.
69. An ingestible particle according to item 68, wherein the binder is selected from hydroxypropyl cellulose (HPC) and combinations thereof.
70. An ingestible particle according to item 68, wherein the polysaccharides are selected from high molecular weight alginates, pectins, gum tragacanth and gum acacia, and combinations thereof.
71. An ingestible particle according to item 67, wherein the binder is selected from calcium carbonate and gum acacia, and combinations thereof.
72. An ingestible particle according to any one of items 56-71, further comprising a lubricant.
73. An ingestible particle according to item 72, wherein the lubricant is selected from stearic acid, magnesium stearate, sodium stearyl fumarate, and combinations thereof.
74. An ingestible particle according to item 73, wherein the lubricant is magnesium stearate.
75. An ingestible particle according to any one of items 56-74, further comprising a glidant.
76. An ingestible particle according to item 75, wherein the glidant is fumed silica.
77. An ingestible particle according to any one of items 56-76, comprising further excipients selected from sugars and complex carbohydrates.
78. An ingestible particle according to item 77, wherein the sugars are selected from glucose, fructose, sucrose and isomaltulose.
79. An ingestible particle according to item 77, wherein the the complex carbohydrates are selected from starch, maltodextrin, dried glucose syrup and dried fructose syrup.
80. An ingestible particle according to any one of items 56 and 59-67, wherein the ingestible particle has the following composition: Sodium bicarbonate 75-85 wt %; Calcium carbonate 5-10 wt %; Hydroxypropyl cellulose 5-10 wt %; Maltodextrin 0.1-1.0 wt %; Gum acacia 1-3 wt %; Magnesium stearate 1-3 wt %; and Anhydrous colloidal silica 0.1-1.0 wt %.
81. An ingestible particle according to any one of items 56 and 59-67, wherein the ingestible particle has the following composition: Sodium bicarbonate 82-92 wt %; Hydroxypropyl cellulose 5-15 wt %; Magnesium stearate 1-3 wt %; and Anhydrous colloidal silica 0.1-1.0%.
82. An ingestible particle according to any one of items 56 and 59-67, wherein the ingestible particle has the following composition: Sodium bicarbonate 80-90 wt %; Calcium phosphate 5-15 wt %; Gum acacia 1-5 wt %; Magnesium stearate 1-3 wt %; and Anhydrous colloidal silica 0.1-1.0 wt %.
83. Use of ingestible particles according to any one of items 56-82 in a nutritional supplement composition to improve high-intensity exercise performance.
84. Use of ingestible particles according to any one of items 56-82 to improve high-intensity exercise performance.
85. Use of ingestible particles according to any one of items 56-82 in a nutritional supplement composition to prevent, alleviate or mitigate exercise-induced acidosis and/or acidemia.
86. Use of ingestible particles according to any one of items 56-82 to prevent, alleviate or mitigate exercise-induced acidosis and/or acidemia.
87. A method for improving high-intensity exercise performance, comprising the step of ingesting the ingestible particles according to any one of items 56-82.
88. A method for preventing, alleviating or mitigating exercise-induced acidosis and/or acidemia in a subject, comprising the step of ingesting the ingestible particles according to any one of items 56-82.
89. A method according to any one of items 87-88, wherein ingestible particles containing a total amount of 0.10-0.40 g bicarbonate salt/kg body mass are thereby ingested; preferably 0.20-0.35 g bicarbonate salt/kg body mass; preferably 0.25-0.30 g bicarbonate salt/kg body mass.
90. A method of treating metabolic acidosis and/or acidemia in a human subject in need thereof, comprising the step of ingesting a pharmaceutically effective amount of the ingestible particles according to any one of items 56-82.
91. A method according to item 90, wherein the metabolic acidosis and/or acidemia is caused by chronic kidney disease.
92. A method according to any one of items 90-91, wherein ingestible particles containing a total amount of 0.01-0.10 g bicarbonate salt/kg body mass are thereby ingested; preferably 0.02-0.07 g bicarbonate salt/kg body mass; preferably 0.02-0.04 g bicarbonate salt/kg body mass.
93. Ingestible particles according to any one of items 56-82 for use as a medicament.
94. Ingestible particles according to any one of items 56-82 for use in a method according to any one of items 90-92.
95. Use of ingestible particles according to any one of items 56-82 in the preparation of a medicament for the treatment of metabolic acidosis and/or acidemia.

1. A nutritional supplement composition which is a suspension comprising ingestible particles comprising sodium bicarbonate, dispersed in an aqueous medium; wherein
the particles are tablets with a thickness of 1.0-2.5 mm and a diameter of 1.0-5.0 mm;
the particles contain more than 50% (w/w) of the sodium bicarbonate;
the aqueous medium is a viscous aqueous medium; and
the sodium bicarbonate content in the ingestible particle is fully dissolved within less than 2 h after ingestion.

2. The nutritional supplement composition according to claim 1, wherein the ingestible particles comprise more than 65% (w/w) of the sodium bicarbonate, such as more than 75% (w/w) of the sodium bicarbonate.

3. The nutritional supplement composition according claim 1, wherein the ingestible particles are suitable for releasing the sodium bicarbonate in the small intestine.

4. The nutritional supplement composition according to claim 1, wherein the tablets have a thickness of 1.0-2.0 mm and a diameter of 1.0-5.0 mm.

5. The nutritional supplement composition according to claim 4, wherein the tablets have a diameter of ≤2.0 mm.

6. The nutritional supplement composition according to claim 1, wherein the ingestible particles have no coating.

7. The nutritional supplement composition according to claim 1, wherein the ingestible particles are homogenous and not layered.

8. The nutritional supplement composition according to claim 1, wherein the ingestible particles are further comprising a binder.

9. The nutritional supplement composition according to claim 8, wherein the binder is selected from hydroxypropyl cellulose (HPC) and combinations thereof.

10. The nutritional supplement composition according to claim 1, wherein the ingestible particles have the following composition:
Sodium bicarbonate 82-92 wt %; Hydroxypropyl cellulose 5-15 wt %;
Magnesium stearate 1-3 wt %; and Anhydrous colloidal silica 0.1-1.0%.

11. The nutritional supplement composition according to claim 1, wherein the sedimentation rate of a single ingestible particle in the unstirred viscous aqueous medium is less than 5 mm/min.

12. The nutritional supplement composition according to claim 1, wherein the viscous aqueous medium is a viscoelastic medium.

13. The nutritional supplement composition according to claim 12, wherein the viscoelastic medium is a gel.

14. The nutritional supplement composition according to claim 1, wherein the viscous aqueous medium comprises one or more natural polymers dissolved in water as a thickener.

15. The nutritional supplement composition according to claim 14, wherein the natural polymers are polysaccharides.

16. The nutritional supplement composition according to claim 15, wherein the polysaccharides are selected from the group consisting of native and modified starch, xanthan gum, guar gum, karragenan, alginate, pectin, and any combination thereof.

17. The nutritional supplement composition according to claim 1, wherein the viscous aqueous medium has the following composition:
4-12 wt % maltodextrin; 3-10 wt % fructose; 2-7 wt % acetylated distarch adipate; and 0.1-1.0 wt % xanthan gum; in water to 100 wt %.

18. The nutritional supplement composition according to claim 1, wherein the viscous aqueous medium has the following composition:
6-14 wt % maltodextrin, 2-5 wt % fructose, 1-3 wt % acetylated distarch adipate and 0.1-1.0 wt % xanthan gum; in water to 100 wt %.

19. A kit for preparing a nutritional supplement composition, the kit comprising ingestible particles and an aqueous medium as defined in claim 1.

20. A method comprising:
ingesting a nutritional supplement composition according to claim 1 before, during or after exercise.
